(19) United States
(12) Patent Application Publication
(10) Pub. No.: US 2009/0298159 A1
(43) Pub. Date: Dec. 3, 2009

(54) Method for producing biodiesel from an alga

(75) Inventors: Qingyu Wu, Beijing (CN); Wei Xiong, Beijing (CN)

Correspondence Address: Carlineo, Spicer & Kee, LLC, 2003 S. Easton Road, Suite 208, Doylestown, PA, 18901, US

(73) Assignee: Tsinghua University

(21) Appl. No.: 12/313,455

(22) Filed: Nov. 20, 2008

(30) Foreign Application Priority Data
May 27, 2008 (CN) 200810112998.9

(51) Int. Cl. C12N 1/12; C07C 69/52

(52) U.S. Cl. 435/257.3; 560/205

(57) ABSTRACT

A method is provided to produce biodiesel from algae using a two-stage, autotrophic and heterotrophic cultivations of chlorella for biodiesel production. This method includes a sequence of procedures: cultivating photoautotrophic algae, concentrating cells and then transferring them to a fermentor for heterotrophic cultivation. During the photoautotrophic cultivation stage, the culture is exposed to a light source, such as sunlight with carbon dioxide obtained from a carbon dioxide source or from air. antibacterial agents may be added to prevent contamination from undesired microorganisms. Organic carbons are added during heterotrophic cultivation stage. Fermentation conditions are optimized for maximizing lipid synthesis. High biomass is achieved to about 108 g/L with lipid content reaching about 52% of dry cell weight. After cultivation, biodiesel is made through extraction and transesterification of algae lipids.

### CROSS REFERENCE TO RELATED APPLICATION

This application claims the benefit of China Patent Application No. 200810112998.9, filed on May 27, 2008, and entitled Method of Two-stages, Autotrophic and Heterotrophic Cultivations of Microalgae for Producing Biodiesel, the entire disclosure of which is incorporated herein by reference.

This application is related to U.S. patent application Ser. No. 12/080,666, filed on April 2, 2008 and entitled Method For Producing Biodiesel Using High-Cell-Density Cultivation Of Microalga Chlorella Protothecoides In Bioreactor, the entire disclosure of which is incorporated herein by reference.

### FIELD OF THE INVENTION

The present invention relates to area of regenerable biofuel, in particular to a method for producing biodiesel using autotrophic and heterotrophic two-stage cultivations of microalgae.

### BACKGROUND OF THE INVENTION

Biodiesel refers to monalkyl esters of long chain fatty acids derived from animal, plant or microorganism lipids which can be used in diesel-engine vehicles. Biodiesel has attracted increasing attention and rapidly developed in large energy-consuming countries because they are renewable and environmental friendly. Currently, economic crops (e.g. soybean, rapeseed, palm, etc.) are the main feedstock of biodiesel in Europe and America. Due to their low oil yield potential and high demands on land, water, and fertilizer, which create competition with food/feed industries, the conventional oil/food crop-based biodiesel production system cannot meet the growing demand on sustainable feedstock for biodiesel production.

Microalgae are a group of photosynthetic microorganism. They are capable of absorbing CO₂ from the atmosphere to synthesize lipids. With further processing, these lipids can be extracted, harvested and refined to be biodiesel used as automobile fuel and jet fuel.

As an ideal alternative of biodiesel feedstock, microalgae, which can grow in salty or polluted water, do not compete for agricultural land and fresh water. Moreover, they reproduce approximately 40 times faster than higher plants with astounding manufacturing capacities.

In aforementioned U.S. patent application Ser. No. 12/080,666, Wu et al. used a heterotrophic cultivation technique to grow microalgae Chlorella protothecoides to achieve high cell densities and high lipid contents, which is suitable for large scale production of biodiesels. Heterotrophic growth of microalgae, however, employing mainly organic carbon as substrates. Some of these organic carbons, such as glucose and starch, also come from conventional agriculture and compete with food/feed industries.

Photoautotrophic cultivation is an environmental friendly way to fix carbon dioxide, the main greenhouse gas, and to release oxygen as a by-product. However, light utilization is often limited with the increase of cell concentration, due to mutual shading between algal cells, which makes it difficult to achieve high-cell-density of oleaginous microalgae in photo-bioreactors.

Therefore, there is a need to further reduce consumptions of organic carbons in heterotrophic cultivation of microalgae and fully use other energy sources, such as sunlight, and inorganic carbons, such as CO₂, to increase biomass of microalgae to produce biodiesels.

### SUMMARY OF THE INVENTION

The present invention provides a novel method to produce oil feedstock for biodiesel production using a two-stage process for growing microalgae photoautotrophically and heterotrophically.

One preferred embodiment of current invention comprises the steps of: growing microalgae by utilizing inorganic carbon (CO₂) and light under autotrophic conditions to accumulate biomass; concentrating the cells; and supplementing the cells with organic carbon to cause the chlorella to accumulate lipids under heterotrophic growing environment.

Another preferred embodiment of current invention comprises the steps of: photoautotrophic cultivating a freshwater green algae, chlorella; concentrating the photoautotrophic cultivated chlorella cells; and heterotrophically fermenting the concentrated photoautotrophic cultivated chlorella cells.

One of aforementioned preferred embodiments of current invention further comprises the steps of: harvesting and drying of the microalgae cells; extracting lipids from the dried microalgae cells; and performing esterification of the extracted lipids to generate a biodiesel.

In another preferred embodiment of current invention, wherein the microalgae is an oleaginous microalgae strain. The oleaginous microalgae strain further comprises Chlorella protothecoides.

In one of aforementioned preferred embodiments of current invention, wherein the autotrophic growing or photoautotrophic cultivating of the algae further comprises placing the algae culture in a flask, a cultivation pond or a photo-bioreactor; exposing the culture to light including but not limiting to sunlight; and controlling a temperature around the culture at between 20-45° C.

In one of aforementioned preferred embodiments of current invention, wherein the autotrophic growing or photoautotrophic cultivating of the algae further comprises controlling initial concentration of glycine in the culture at between 1-15 g/L and pH at between 5-9; controlling air circulation rate between 50-300 L/h and CO₂ concentration between 0.9-3%; and using 25-200 μmol.m⁻²s⁻¹ of light.

In one of aforementioned preferred embodiments of current invention, wherein the heterotrophic growing or heterotrophically fermenting of the concentrated algae cells further comprises: controlling an initial cell density between 5-100 g/L, temperature of 15-45° C., air flow rate of 100-300 L/h; adding acid or base to control the pH to be between 5.0-9.0; and adding organic carbon to maintain the sugar concentration of the growth or fermenting system between 1-40 g/L.

In one of aforementioned preferred embodiments of current invention, wherein the heterotrophic growing or heterotrophically fermenting of the concentrated algae cells further comprises: controlling a stirring rate to maintain dissolved oxygen above 10% in the growth or fermenting system; adding chloramphenicol or MFA to prevent possible microbial contamination during autotrophic cultivation and cell concentration processes; and maintaining the concentration of chloramphenicol between 0.002-0.2 g/L, or the concentration of MFA between 0.1-100 mM.

One of aforementioned preferred embodiments of current invention further an autotrophic growing or photoautotrophically cultivating medium. One of the preferred composition of the autotrophic growing or photoautotrophically cultivating medium further comprises: KH2PO4; K2HPO4; MgSO4.7H2O; FeSO4.7H2O; glycine; vitamin B1; and A5 trace element liquid; where the A5 trace element liquid contains: H3BO3; Na2MoO4.2H2O; ZnSO4.7H2O; MnCl2.4H2O; and CuSO4.5H2O.

One of aforementioned preferred embodiments of current invention further an heterotrophically cultivating or heterotrophically fermenting medium. One of the preferred composition of the heterotrophically cultivating or heterotrophically fermenting medium further comprises: KH2PO4; K2HPO4; MgSO4.7H2O; FeSO4.7H2O; vitamin B1; and A5 trace element liquid; where the A5 trace element liquid contains: H3BO3; Na2MoO4.2H2O; ZnSO4.7H2O; MnCl2.4H2O; and CuSO4.5H2O.

In one of aforementioned preferred embodiments of current invention, wherein the heterotrophic growing or heterotrophically fermenting of the concentrated algae cells further comprises: adding organic carbon until the initial reducing sugar concentration reaches 0.01-100 g/L; and maintaining the concentration of reducing sugar between 1-30 g/L.

In one of aforementioned preferred embodiments of current invention, wherein the reducing sugar includes but not limit to glucose or other monosaccharides, disaccharides or polysaccharides, the source of organic carbon comprises glucose, fructose, corn starch hydrolysate, cassava starch hydrolysate, wheat starch hydrolysate, or sorghum juice.

Other objects, advantages, and features of the present invention will become apparent from the following detailed description of the invention.

### BRIEF DESCRIPTION OF THE DRAWINGS

The following detailed description will be better understood when read in conjunction with the appended drawings, in which there is shown one or more of the multiple embodiments of the present invention. It should be understood, however, that the various embodiments of the present invention are not limited to the precise arrangements and instrumentalities shown in the drawings.

In the Drawings:

FIG. 1 is a process flow schematic for biodiesel production using autotrophic to heterotrophic two-stage cultivations of chlorella;

FIG. 2 is an autotrophic growth curve of chlorella cells in a photo-bioreactor;

FIG. 3 is a growth curve and glucose consumption of algae cells in a 5 L bioreactor after conversion to heterotrophic cultivation;

FIG. 4A is a fluorescence spectrum of Nile Red-stained cells, showing the profile of autotrophic chlorella; and

FIG. 4B is a fluorescence spectrum of Nile-Red-stained cells showing the results of chlorella after conversion to heterotrophic cultivation.

### DETAILED DESCRIPTION

Certain terminology is used herein for convenience only and is not to be taken as a limitation on the embodiments of the present invention. It should be appreciated that the particular embodiments shown and described herein are examples of the invention and are not intended to otherwise limit the scope of the present invention in any way.

One objective of the present invention is to provide a novel method of biodiesel production using a two-stage process for growing microalgae photoautotrophically and heterotrophically. Biodiesels are made from heterotrophic algae by cultivating photoautotrophic algae, concentrating cells and preventing undesired bacterial contamination. One preferred approach is to allow microalgae chlorella grow autotrophically, leading the chlorella cells to accumulate biomass rapidly, then allow the chlorella cells to grow heterotrophically to synthesize lipids under high cell density conditions.

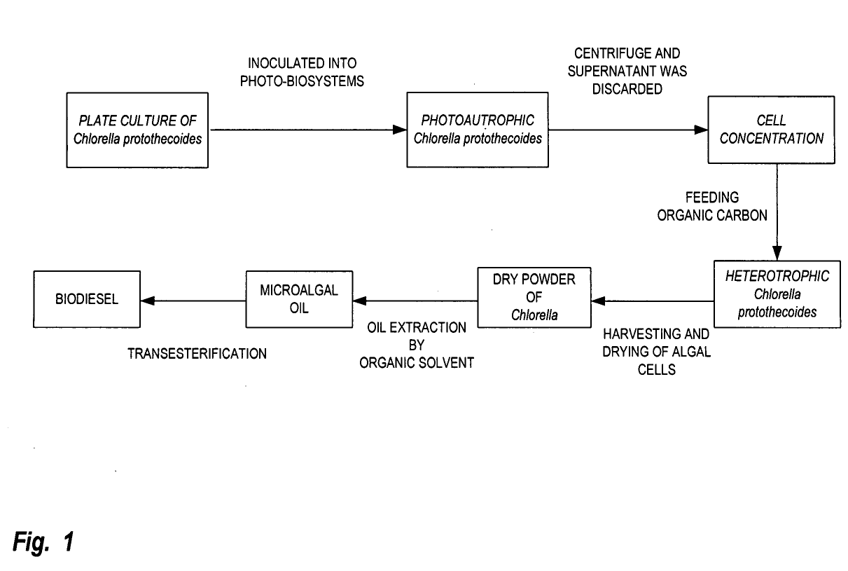

Referring generally to FIG. 1, a preferred embodiment of the present invention is illustrated:

### (1) Photoautotrophic Cultivation of Chlorella

Plate culture of freshwater green algae Chlorella protothecoides is inoculated into a photosynthetic apparatus to grow with light in an autotrophic medium, wherein the photosynthetic apparatus can be a glass flask, a photo-bioreactor or an open pond. Culture conditions are set as follows: temperature controlled between 20-45° C., preferably at about 30° C.; initial concentration of glycine between 1-15 g/L, preferably at about 5 g/L; pH controlled between 5-9, preferably at about 6.5; inflow of air or a mixture comprising air and CO₂ at a rate of 50-300 L/h, preferably at about 80-120 L/h; a CO₂ concentration of 0.9-3%. The amount of sunlight used during the cultivation process is 25-200 μmol.m⁻²s⁻¹. The total cultivation time depends on the cell growth situation, and is generally between 50-400 hours, preferably between 120-200 hours.

The above discussed autotrophic cultivation medium comprises:
KH₂PO₄: 0.2-0.7 g/L,
K₂HPO₄: 0.1-0.4.3 g/L,
MgSO₄.7H2O: 0.1-0.4 g/L,
FeSO₄.7H₂O: 0.1-3 mg/L,
Glycine: 0.1-15 g/L,
vitamin B₁: 0.001-0.1 mg/L, and
A5 trace mineral solution 1.0 ml/L, wherein the A5 trace mineral solution comprises H₃BO₃, Na₂MoO₄.2H₂O, ZnSO₄.7H₂O, MnCl₂.4H₂O, and CuSO₄.5H₂O.

A preferred A5 trace mineral solution comprises:
H₃BO₃: 2.86 g/L,
Na₂MoO₄.2H₂O: 0.039 g/L,
ZnSO₄.7H₂O: 0.222 g/L,
MnCl₂.4H₂O: 1.81 g/L, and
CuSO₄.5H₂O: 0.074 g/L.

### (2) Concentrating the Cells

Many techniques, including but not limited to, high-speed centrifugation, flocculation or filtering technology can be used to concentrate cells. An example for such a concentration is to use a high-speed centrifugation in microorganism-free conditions. A centrifugal force, being set as 2000-8000 g, applies to the photoautotrophically cultivated culture eat 4° C. for about 2-5 min. Autotrophic cell pellet can be harvested by discarding the supernatant.

### (3) Heterotrophic Fermentation

The concentrated cells are put into a bioreactor, which includes but not being limited to a reaction vessel and a pond. In the process of converting chlorella from autotrophic to heterotrophic growth, the growth characteristics of the cells and their biochemical composition both undergo obvious changes: the chlorophyll content decreases, the thylakoids disappear, and the average cell weight decreases. Lipid synthesis pathways for carbon flow are induced by adding organic carbon. A preferred process for high-cell-density heterotrophic cultivation coupled with efficient synthesis of lipids are set as follows:

The autotrophically grown cells collected are re-suspended in sterile medium containing organic carbon, making the initial density of the cells between 5-100 g.L/1. The organic carbon may comprise glucose, fructose, hydrolysates of corn, cassava, or wheat starches, sorghum juice etc. with the reducing sugar concentration chosen between 0.01-100 g/L, preferably at about 23 g/L.

The heterotrophic cultivation medium comprises:
KH₂PO₄: 0.2-0.7 g/L,
K₂HPO₄: 0.1-0.4.3 g/L,
MgSO₄.7H₂O: 0.1-0.4 g/L,
FeSO₄.7H₂O: 0.1-3 mg/L, and
A5 trace mineral solution 1.0 ml/L;
   wherein organic carbons are added to maintain the initial sugar concentration between 0.01-100 g/L. During heterotrophic cultivation, organic carbon is continuously added to maintain the concentration between 1-30 g/L.

Heterotrophic process is performed in a fermentation vessel equipped with installed electrodes of temperature, pH, and Dissolved Oxygen (DO) measurements. Culture medium is added into the fermentation vessel for sterilization. One set of preferred fermentation conditions are set as follows:
(a) the pressure in the vessel, air flow rate and stirring speed are adjusted to make the initial oxygen saturation at 100%;
(b) temperature is set between 15-45° C., preferably at about 29° C.;
(c) rate of air flow between 100-300 L/h; and
(d) Acid or base (KOH, H2SO4 etc) is added to maintain the pH of the system between 5.0-9.0.

During the fermentation process, stirring speed is controlled to maintain the oxygen saturation level greater than 10%. Organic carbon is fed to keep the reducing sugar concentration between 1-40 g/L. Samples are taken regularly to determine the cell density and sugar concentration in the medium. Nile Red, a fluorescent dye to detect neutral lipid, is also utilized. Fermentation is preferably terminated when the cell density exceeds 100 g.L/1 and lipid content reaches 50% of the cells' dry weight. A typical fermentation process takes about 50 to about 300 hours.

To prevent possible contamination by undesired microorganisms during autotrophic cultivation and cell concentrating processes, antibacterial agents are added to inhibit microorganisms' growth. The antibacterial agents may include but not be limited to, chloramphenicol or monoflouroacetate (MFA). The concentration of chloramphenicol in the medium is between 0.002-0.2 g/L, preferably at about 0.01 g/L. When monoflouroacetate is used, the concentration of monoflouroacetate is between 0.1-100 mM, preferably at about 2 mM.

### (4) Harvesting and Drying of Algae Cells

Many solid-liquid phase separation technologies can be employed to harvest algae cells from the fermented medium. This process includes but not being limited to, precipitation, filtration, centrifugation and drying. Algae cells obtained may be stored as dry powder. Many drying techniques may be used, including but not limiting to, freeze-drying, spray drying, and sunlight drying.

(5) Extraction of Lipids from Dried Algae Cells

The method of extracting lipids from the dried algae cells may include and not limiting to high pressure solvent extraction or Soxhlet extraction. When Soxhlet extraction is used, hexane is employed as the standard extraction solvent. The lipids are separated from the algae powder by washing repeatedly with hexane. The solvent can be removed by a reduced pressure distillation.

### (6) Generating Biodiesel by Esterification

Biodiesels can be made from the algal lipids by techniques including but not limiting to, esterification. The conversion from fatty acids to esters of fatty acids includes, but is not limited to the process catalyzed by acid, such as concentrated sulfuric acid, or lipase. The fatty acid methyl esters resulted from this catalyzed process forms the main component of biodiesel.

In summary, present invention provides a technical route to produce microalgal biodiesel through two-stage (first autotrophic then heterotrophic) cultivation of Chlorella. The two-stage process takes advantage of separating cell factories construction (photoautotrophic microalgae growth) and products manufacture (lipid synthesis), wherein each of the stage can be optimized respectively. A cell concentrating step to bridge these two stages to efficiently use each of these stages to achieve high light energy conversion efficiency, high lipid contents and low glucose consumption. Carbon dioxide in the environment can be fixed to the autotrophic culture, while the carbon dioxide emission in the heterotrophic culture is reduced significantly. Final algae cell density (unit yield) can be reached to 108 g/L (dry weight), and cell lipid contents are over 50%.

### EXAMPLE

### Materials and Methods

Chlorella protothecoides, an oleaginous microalgae strain was purchased from Culture Collection of Algae in University of Texas (Austin, Tex.), which culture has been reserved in the Algal Bio-Energy Lab of the Department of Biological Science and Biotechnology at Tsinghua University for bio-energy research. The autotrophic medium for this strain has the following composition:
KH₂PO₄: 0.6 g/L,
K₂HPO₄: 0.4 g/L,
MgSO₄.7H₂O: 0.4 g/L,
FeSO₄.7H₂O: 1 mg/L,
Glycine: 3 g/L,
vitamin B₁: 0.03 mg/L, and
A5 trace mineral solution 1.5 ml/L, where the A5 trace element liquid contains H₃BO₃: 2.86 g/L; Na₂MoO₄.2H₂O : 0.039 g/L; ZnSO₄.7H₂O: 0.222 g/L; MnCl₂.4H₂O: 1.81 g/L; CuSO₄.5H₂O : 0.074 g/L.

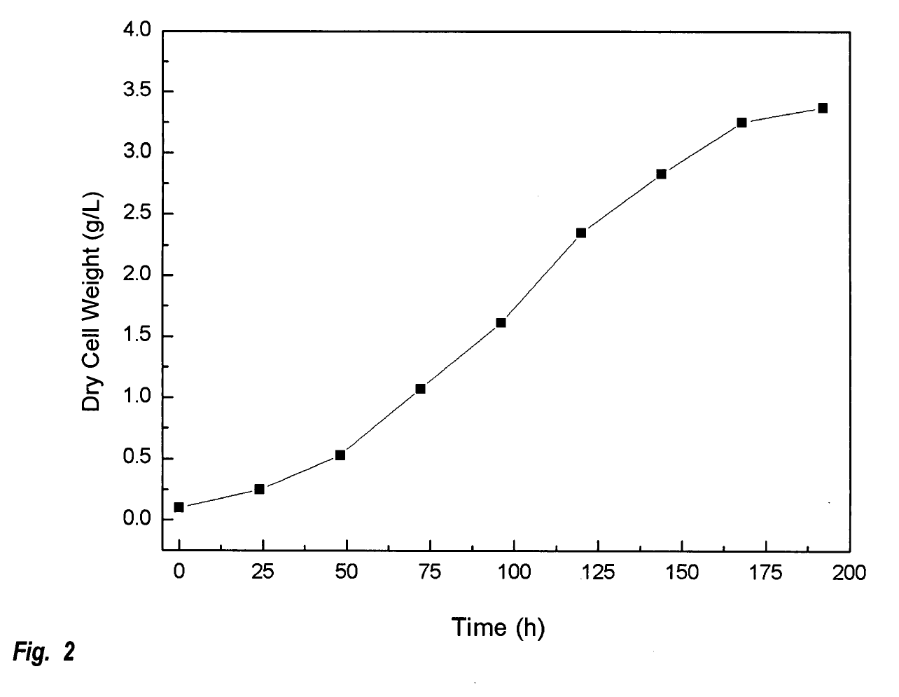

As illustrated in FIG. 1, a colony of chlorella grown on the agar plate is inoculated to a glass flask with an initial cell density 0.1 g/L. The flask is placed in an illumination incubator at 25° C. with circulating sterile air at a rate of 100 L/h. The pH of medium is maintained at about 5 to about 9 for autotrophic cultivation. Various cultivation conditions, such as nitrogen sources, illumination intensity were monitored. The concentration of glycine in the medium is set to be in a range from about 1 g/L to about 9 g/L. Light intensities is set to be in a range of about 25 to about 100 μmol.m⁻²s⁻¹. Preferred conditions of glycine concentration and light intensity for autotrophic cultivation under the above temperature and air circulation rate, are about 5 g/L and 100 μmol.m⁻²s⁻¹, respectively. Cell growth under such preferred conditions are illustrated in FIG. 2.

When the cell growth enters the late stage of the log phase, the air circulation (bubbling) is stopped to allow the cells to settle to the bottom of the flask for 12 hours. After supernatant liquid is discarded, the remaining liquid is further removed by centrifuge at 3000 g for about 2 minutes.

A 5 L fermentor (MINIFORS, Switzerland) equipped with temperature, pH, DO electrodes is used. A heterotrophic medium is added into the fermentor, which is sterilized at 121° C. for about 30 minutes. The heterotrophic medium composition is as follows:
KH2PO4: 0.6 g/L
K₂HPO₄: 0.4 g/L,
MgSO₄.7H₂O: 0.4 g/L
FeSO₄.7H2O: 0.5 mg/L
vitamin B1: 0.08 mg/L
A5 trace element liquid 1.5 ml/L
glucose 23 g/L, and
monoflouroacetate (MFA) at a final concentration of 1 mM was added in order to prevent microbial contamination.

Conditions for heterotrophic cultivations are set as follows:
temperature maintained at about 29° C.,
air flow rate set at about 150 L/h,
pH adjusted to 6.2±0.2 through addition of acid or base (e.g. KOH, H2SO4, etc), and
dissolved oxygen maintained above 10% of the saturation.

Stirring speed is increased when DO value drops below 10%. When all carbon is consumed and DO value suddenly increases, glucose is added to control the concentration of glucose between 1-30 g/L. A fed-batch process control can be employed to achieve high cell density cultivation by monitoring changes in DO value, glucose concentration, cell density and neutral lipid content of the algae cells in the fermentor.

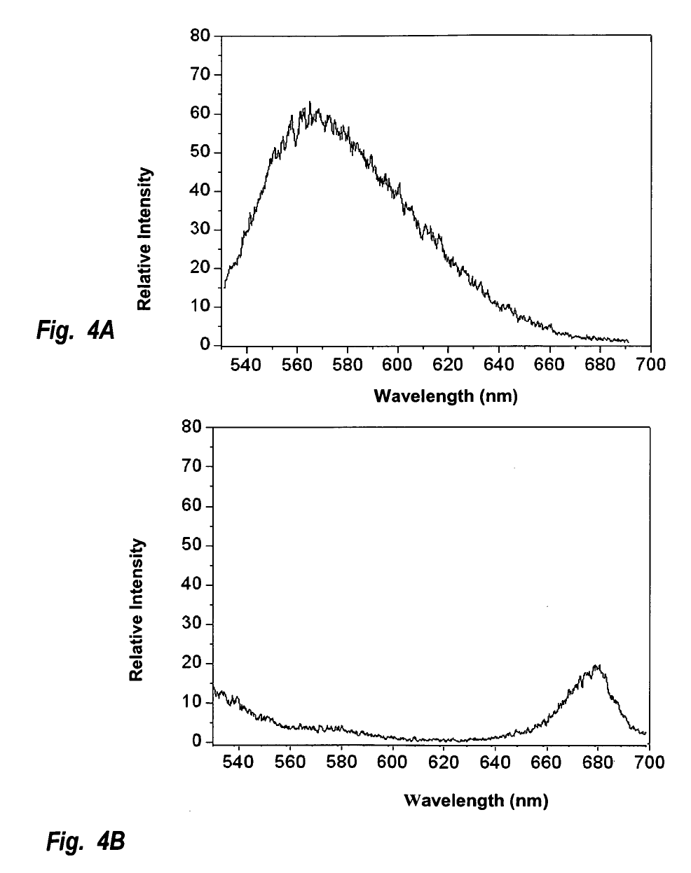

The neutral lipid content is measured using Nile Red mediated fluorescence technique (specific procedures described in “Danielle E, David J, Barry R, et al. 2007. Fluorescent measurement of microalgal neutral lipids. Journal of Microbiological Methods, 68 (3): 639-642.”). FIG. 4A illustrates fluorescent spectrum of autotrophically grew chlorella, and FIG. 4B shows fluorescent spectrum of the Chlorella after heterotrophic cultivation, wherein the emission peak for chlorophyll is 680 nm while the Nile Red emission peak is 570 nm. FIGS. 4A and 4B illustrate strong photosynthesis occurs during the autotrophic cultivation stage, while significant lipid generation occurs during the heterotrophic cultivation stage. Current invention takes advantages of both autotrophic and heterotrophic cultivations to achieve high lipid yield with low consumption of organic carbons for making biodiesels.

The cell density during the fermentation process is estimated by periodic measurements of optical intensity (OD540 nm). The linear relationship between OD540 nm and the dry weight of the cells may be expressed by the following equation:

y=0.4155x, (R2=0.9933, P<0.05),

wherein y represents cell density (g/L, and x represents the optical intensity at 540 nm.

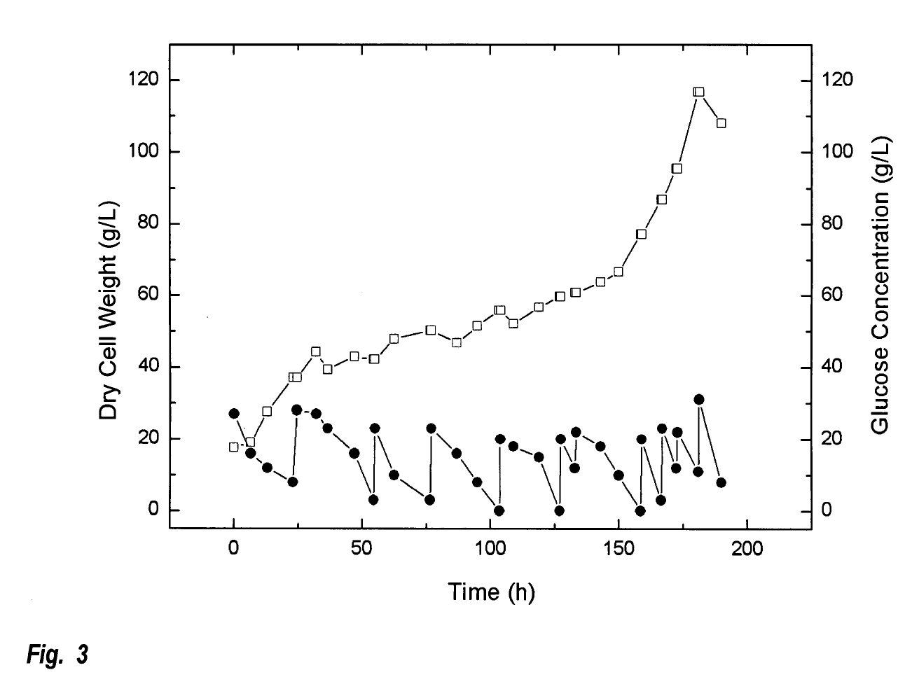

After fermentation, cells are harvested from the fermentation culture using centrifugation at about 8000 g for 2 minutes, flowed by freezing drying the pellet in vacuum. An exemplary dry algae powder yielded, 108 g/L, is illustrated in FIG. 3.

Lipids can be extracted from the algae powder using a Soxhlet extractor, a semiautomatic solvent extraction device with hexane as the standard solvent. The dried power is washed repeatedly with hexane. Molecular weights of the lipids from heterotrophically cultivated algae can be calculated as follows:

Molecular weight (M)=56.1×1000×3/(SV−AV),

where SV represents the saponification value, and AV represents the acid value of the algae lipids, the resulting molecular mass value calculated is a dimensionless number.

Rotary evaporation can be used to remove the solvent. The yield of lipids by Soxhlet extraction is in about 52% (w/w) lipids in the cells. The total consumption of glucose over the cultivation process was 280 g/L, which leads the lipid yield as 56.16 g/L (108 g/L×52%) and a conversion rate of glucose to lipid as 20.06%. The two-stage cultivation process can effectively lower organic carbon consumption, comparing the conversion rate of glucose to lipid of 10% in a high cell density cultivation process.

Esterification of the extracted lipids can be catalyzed using sulfuric acid. In a preferred embodiment, equal quantities of the extracted algae lipids and concentrated sulfuric acid, e.g. 90% sulfuric acid solution, are placed in a container, such as a flask. Methanol solvent s is added into the container to maintain the molar ratio of methanol to lipid to be 30:1. The catalyzed esterification is carried out at 55° C. with a stirring rate at 160 RPM for 48 hours. After completion of the esterification, reaction mixture is separated into two layers. The upper layer contains biodiesel mixing with solvent, which is washed with warm water at 30° C., and then rotary evaporated to obtain biodiesel.

Contents of tri-, di-, and monoglycerides, methanol, and glycerol of the obtained biodiesel can be analyzed using gas chromatography—mass spectrometry (GC-MS). DSQ GC (Thermo, USA, VARIAN VF-5 ms Capillary 30M*0.25MM) gas chromatography—mass spectrometry is employed, with flow rate of 10 ml/min. Operation temperature of the GC-MS is set as follows: temperature is first raised to 70° C. and maintained at 70° C. for 2 minutes; then the temperature is raised to 300° C. at the rate of 10° C./min and maintained at 300° C. for 20 minutes. Temperature of inject entrance is set as 250° C. with flow ratio of 30:1. The catalyzed esterification can convert over 90% of the algae lipids to fatty acid methyl esters (biodiesel).

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of the present invention is not limited to the particular examples and implementations disclosed herein, but is intended to cover modifications within the spirit and scope thereof as defined by the appended claims and any and all equivalents thereof.

What is claimed is:

1. A method for producing biodiesel from an alga, the method comprising:
autotrophically cultivating said alga;
concentrating said autotrophically cultivated alga;
heterotrophically cultivating said concentrated alga;
collecting and drying said heterotrophically cultivated alga;
extracting said dried alga; and
performing esterification of said extracted dried alga to produce said biodiesel.

2. The method of claim 1, wherein autotrophically cultivating said alga further comprises photoautotrophically cultivating said alga.

3. The method of claim 2, wherein said photoautotrophically cultivating said alga further comprises inducing carbon dioxide or carbon dioxide containing gas into a culture wherein said photoautotrophically cultivating said alga occurs.

4. The method of claim 1, wherein autotrophically cultivating said alga further comprises an autotrophic cultivation medium wherein said autotrophic cultivation medium further comprises KH2PO4; K2HPO4; MgSO4.7H2O; FeSO4.7H2O; glycine; vitamin B1; and A5 trace element liquid; wherein said A5 trace element liquid contains: H3BO3; Na2MoO4.2H2O; ZnSO4.7H2O; MnCl2.4H2O; and CuSO4.5H2O.

5. The method of claim 1, wherein heterotrophically cultivating said concentrated alga further comprises an heterotrophic cultivation medium wherein said autotrophic cultivation medium further comprises KH2PO4; K2HPO4; MgSO4.7H2O; FeSO4.7H2O; vitamin B1; and A5 trace element liquid; wherein said A5 trace element liquid contains: H3BO3; Na2MoO4.2H2O; ZnSO4.7H2O; MnCl2.4H2O; and CuSO4.5H2O.

6. The method of claim 5, further comprising adding organic carbon into said heterotrophic cultivation medium.

7. The method of claim 6, wherein said organic carbon further comprises reducing sugar including glucose or other monosaccharides, disaccharides or polysaccharides.

8. The method of claim 6, wherein said organic carbon further comprises glucose, fructose, corn starch hydrolysate, cassava starch hydrolysate, wheat starch hydrolysate, or sorghum juice.

9. The method of claim 7, wherein the concentration of the reducing sugar of said heterotrophic cultivation medium is maintained at about 1 g/L to about 30 g/L during the step of said heterotrophically cultivating said concentrated alga.

10. The method of claim 1, wherein autotrophically cultivating said alga further comprises adding an antibacterial agent.

11. The method of claim 10, wherein said antibacterial agent further comprises chloramphenicol.

12. The method of claim 11, wherein the concentration of said chloramphenicol is maintain at about 0.002 g/L to about 0.2 g/L.

13. The method of claim 10, wherein said antibacterial agent further comprises monoflouroacetate (MFA).

14. The method of claim 13, wherein the concentration of said MFA is maintained at about 0.1 to about 100 mM.

15. The method of claim 1, wherein autotrophically cultivating said alga further comprises maintaining a temperature at about 20 to about 45° C.

16. The method of claim 3, wherein d autotrophically cultivating said alga further comprises maintaining the concentration of carbon dioxide in said culture at about 0.9% to about 3%.

17. The method of claim 1, wherein autotrophically cultivating said alga occurs under exposure of sunlight.

18. The method of claim 1, wherein a pH is maintained at about 5 to about 9 by adding a base or an acid in the step of said autotrophically cultivating said alga or in the step of said heterotrophically cultivating said concentrated alga.

19. A method for cultivating an alga, the method comprising:
autotrophically cultivating said alga;
concentrating said autotrophically cultivated alga; and
heterotrophically cultivating said concentrated alga;
wherein said alga is Chlorella.

20. The method of claim 19, further comprising adding carbon dioxide or carbon dioxide containing gas and exposing to sunlight autotrophically cultivating said alga; and adding an organic carbon in said heterotrophically cultivating said concentrated alga.
